(19) United States
(12) Patent Application Publication
(10) Pub. No.: US 2015/0037842 A1
(43) Pub. Date: Feb. 5, 2015

(54) MODIFIED BACTERIAL CELL

(71) Applicant: The University of Warwick, Coventry (GB)

(72) Inventor: Colin Robinson, Coventry (GB)

(21) Appl. No.: 14/382,084

(22) PCT Filed: Feb. 22, 2013
(86) PCT No.: PCT/GB2013/050431

§ 371 Date: Aug. 29, 2014

(30) Foreign Application Priority Data
Mar. 1, 2012 (GB) 1203587.9

(51) Int. Cl. C12N 15/70; C12P 21/00

(52) U.S. Cl. CPC C12N 15/70; C12P 21/00
USPC 435/69.3; 435/252.3; 435/252.33; 435/289.1; 435/69.1; 435/183; 435/69.4; 435/69.52; 435/69.5; 435/69.51; 435/69.6; 435/219; 435/216; 435/213; 435/226; 435/209; 435/208; 435/201; 435/189; 435/229; 435/186; 435/228; 435/191; 435/198; 435/196; 435/199; 435/194

(57) ABSTRACT

The disclosure relates to a Gram negative bacterial cell that is transformed with a nucleic acid molecule that encodes a Gram positive twin-arginine translocase and including methods for the production of polypeptides.

The disclosure relates to a Gram-negative bacterial cell that is transformed with a nucleic acid molecule that encodes a twin-arginine translocase [Tat] from a Gram-positive bacterium and includes methods for the production of recombinant polypeptides.

The large scale production of recombinant proteins [also referred to as heterologous proteins], for example enzymes, polypeptide hormones and monoclonal antibodies, requires a high standard of quality control since many of these proteins are administered to humans. Moreover, the development of vaccines, particularly subunit vaccines, requires the production of large amounts of pure protein free from contaminating antigens which may provoke anaphylaxis. The production of recombinant protein in cell expression systems is based either on prokaryotic cell expression or eukaryotic cell expression. Recombinant polypeptides also include commercially important polypeptides, for example enzymes used in bio-catalysis (e.g. restriction enzymes, enzymes used in industrial processing; e.g. cellulases, amylases, proteases, nucleases, lipases). The ability to secrete polypeptides into the growth medium offers an opportunity-to purify polypeptides without the need for extraction from a host cell expressing said polypeptide.

Bacterial expression systems which produce molecules, in particular peptides and polypeptides, are well known in the art. Typically, bacterial host cells are transformed with a vector that contains expression signals operably linked to a nucleic acid molecule encoding a desired polypeptide sequence. Vectors also have replication origins that facilitate the replication of the vector inside the host bacterium. Gram-positive and Gram-negative bacteria differ in many respects from one another. Gram-negative bacteria are bounded by two separate membranes which are separated by a soluble compartment known as the periplasm. Gram-positive bacteria are bounded by a single membrane. A difference also exists in the nature of their respective cell walls. The biochemical composition of the B. subtilis (Gram-positive) cell wall is quite different from that of E. coli (Gram-negative). The cell walls of Escherichia coli and Bacillus subtilis contain a framework that is composed of peptidoglycan, a complex of polysaccharide chains covalently cross-linked by peptide chains. This forms a semi-rigid structure that confers physical protection to the cell since the bacteria have a high internal osmotic pressure and can be exposed to variations in external osmolarity. In Gram-positive bacteria, such as the members of the genus Bacillus, the peptidoglycan framework may represent as little as 50% of the cell wall complex and these bacteria are characterised by having a cell wall that is rich in accessory polymers such as teichoic acids.

It is known that Gram-negative bacteria do not readily secrete polypeptides into the surrounding growth medium although Gram-positive bacteria do have cell transport mechanisms to secrete polypeptides, these secreted polypeptides can be endogenous polypeptides, (e.g. amylases) or recombinant polypeptides.

The general secretory pathway [Sec] recognizes polypeptide substrates bearing cleavable N-terminal signal peptides and transports them across the inner (plasma) membrane via a membrane-bound translocase in an unfolded form (for reviews see Robinson and Bolhuis, 2004; Müller and Klösgen, 2005). However, despite extensive studies, the Sec machinery cannot efficiently export some proteins of biotechnological or biomedical interest. The twin-arginine translocation (Tat) system offers an alternative to the Sec pathway. It operates in parallel with the Sec pathway in most bacteria but uses a completely different translocation mechanism. As with Sec substrates, Tat substrates are synthesised with N-terminal signal peptides, but these contain specific determinants including the presence of a highly conserved twin-arginine motif.

In Gram-negative bacteria, the Tat system usually comprises 3 proteins, termed TatABC, which are often encoded by an operon (Müller and Klösgen, 2005). In contrast, the vast majority of Gram-positive bacteria possess only TatA and TatC subunits. There are two distinct Tat translocases in Bacillus subtilis, namely TatAdCd and TatAyCy, which are expressed under different growth conditions and which display different substrate specificities (Jongbloed et al., 2004).

This disclosure relates to the expression of a tat operon from a Gram-positive bacterium in a Gram-negative host bacterial cell in combination with the expression of a nucleic acid encoding a polypeptide product that is adapted for export via the Tat system. Production of recombinant proteins in Gram-negative bacteria, particularly Escherichia coli, often involves transporting the protein to the periplasm. The Tat system normally transports proteins into the periplasm. Expression of a Tat system from a Gram-positive bacterium results in the initial targeting of proteins to the periplasm, and the subsequent leakage of periplasmic protein into the growth medium. Further, proteins exported through the TatAdCd system first fold into their native conformation in the cytoplasm and are then exported across the cytoplasmic membrane. This remarkable ability of exporting fully folded proteins is a highly desirable feature for protein production of biotechnological interest. Firstly, proteins that fold prematurely in the cytoplasm or proteins that are unable to fold correctly in the periplasm can be used as Tat machinery substrates. Secondly, there is good evidence that only fully folded proteins are exported by the Tat system's quality control to the periplasm. Finally, because E. coli does not export high amounts of proteins, recovery of a protein from the periplasm is simplified, cytoplasmic contaminations minimized and downstream processing is simplified.

### STATEMENTS OF INVENTION

According to an aspect of the invention there is provided a Gram-negative bacterial cell wherein said cell is genetically modified by transformation with a nucleic acid molecule encoding a polypeptide complex comprising a twin-arginine translocase [Tat] isolated or made from a Gram-positive bacterial species and further wherein said Gram-negative bacterial cell expresses a recombinant polypeptide which polypeptide is adapted to interact with said Tat complex and is secreted into the cell culture medium.

According to an alternative aspect of the invention there is provided a Gram negative bacterial cell wherein said cell is genetically modified by transformation with a nucleic acid molecule encoding a polypeptide complex comprising a twin-arginine translocase [Tat] isolated or made from a Gram positive bacterial species and further wherein said bacterial cell expresses a polypeptide which is adapted to interact with said Tat complex and is secreted into the cell culture medium.

Reference herein to a twin-arginine translocase isolated or made from a gram positive bacterial species is reference to the naturally occurring proteins or their synthetic counterpart, respectively.

In a preferred embodiment of the invention said Tat is over-expressed when compared to a non-transformed reference bacterial cell of the same species expressing reference protein.

In a further preferred embodiment of the invention said cell over-expresses said Tat by at least two-fold when compared to a non-transformed reference bacterial cell of the same species. Preferably said Tat and/or its activity is over-expressed at least 3-fold; 4-fold; 5-fold; 6-fold; 7-fold; 8-fold; 9-fold; or at least 10-fold. More preferably said Tat and/or its activity is over-expressed at least 20-fold; 30-fold; 40-fold; or at least 50-fold. Preferably said tat and/or its activity is over-expressed by at least 100-fold.

The over-expression of Tat and/or its activity can be achieved by means known to those skilled in the art. For example, placing the operon encoding the Tat with the activity of the Tat operon on a high copy number plasmid. Alternatively, or in addition, said gene can be operably linked to a promoter sequence which provides for high level expression of said gene, said promoter can be constitutively active or inducible. Adaptations also include the provision of selectable markers which select for cells containing high copy plasmids. These adaptations are well known in the art. There is a significant amount of published literature with respect to expression vector construction and recombinant DNA techniques in general.

In a preferred embodiment of the invention said Tat comprises the polypeptides TatAdCd.

In an alternative preferred embodiment of the invention said Tat comprises the polypeptides TatAyCy.

In a preferred embodiment of the invention said adaptation is the provision of a signal peptide that interacts with the Tat system, thus, ideally, a protein to be expressed and transported via the Tat system comprises a signal peptide that enables this to happen.

In a preferred embodiment of the invention said signal peptide comprises a twin arginine motif sequence, a hydrophobic domain and a consensus amino acid sequence for a peptidase.

In a preferred embodiment of the invention said consensus motif comprises the amino acid motif RRxFL wherein X is any amino acid residue.

In a preferred embodiment of the invention said signal peptide comprises the amino acid sequence:

(SEQ ID NO: 12)
MANNNDLFQASRRRFLAQLGGLTVAGMLGPSLLTPRRATA.

In a preferred embodiment of the invention said signal peptide consists essentially of the amino acid sequence:

(SEQ ID NO: 12)
MANNNDLFQASRRRFLAQLGGLTVAGMLGPSLLTPRRATA.

In a preferred embodiment of the invention said nucleic acid molecule is selected from the group consisting of:
i) a nucleotide sequence as represented by SEQ ID NO: 1 and 2 or 3 and 4 (in either case with or without the illustrated tag sequence);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the nucleic acid sequences in i) and ii) above wherein said nucleic acid molecule encodes a twin arginine translocase;
iv) a nucleotide sequence that encodes a twin arginine translocase comprising an amino acid sequence represented by SEQ ID NO: 5 and 6 or SEQ ID NO: 7 and 8 (in either case with or without the illustrated tag sequence);
v) a nucleotide sequence that encodes a polypeptide comprising an amino acid sequence wherein said amino acid sequence is modified by addition deletion or substitution of at least one amino acid residue as represented in iv) above and which has retained or enhanced twin arginine translocase activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

A modified polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In one embodiment, the variant polypeptides have at least 50% identity, more preferably at least 50% identity, even more preferably at least 55% identity, still more preferably at least 60%, 65%, 70%, 75%, 80%, 85%, 90%, 95% identity, and at least 99% identity with the full length amino acid sequence illustrated herein.

In a preferred embodiment of the invention said nucleic acid molecule comprises or consists of a nucleotide sequence as represented in SEQ ID NO: 1 and 2.

In a preferred embodiment of the invention said nucleic acid molecule comprises or consists of a nucleotide sequence as represented in SEQ ID NO: 3 and 4.

In a preferred embodiment of the invention said nucleic acid molecule encodes a twin arginine translocase comprising amino acid sequences as represented in SEQ ID NO: 5 and 6.

In a preferred embodiment of the invention said nucleic acid molecule encodes an twin arginine translocase comprising amino acid sequences as represented in SEQ ID NO: 7 and 8.

In a further preferred embodiment of the invention said bacterial cell is a Gram negative bacterial cell, for example Escherichia coli.

Surprisingly, we have found that the transformed cells of the invention possessed novel and potentially superior polypeptide production properties because they transported polypeptide produced thereby into the cell culture medium; upon further investigation we discovered this was due to advantageous leakage from the outer membrane.

According to a further aspect of the invention there is provided a bacterial cell culture comprising a bacterial cell according to the invention.

According to a further aspect of the invention there is provided a cell culture vessel comprising a bacterial cell culture according to the invention.

In a preferred embodiment of the invention said cell culture vessel is a fermentor.

Bacterial cultures used in a process according to the invention are grown or cultured in the manner with which the skilled worker is familiar, depending on the host organism. As a rule, bacteria are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the bacteria as described above by processes known to the skilled worker, for example by extraction, distillation, crystallization, if appropriate precipitation with salt, and/or chromatography. In this process, the pH value is advantageously kept between pH 4 and 12, preferably between pH 6 and 9, especially preferably between pH 7 and 8.

An overview of known cultivation methods can be found in the textbook by Chmiel (Bioprozeβtechnik 1. Einführung in die Bioverfahrenstechnik [Bioprocess technology 1. Introduction to Bioprocess technology] (Gustav Fischer Verlag, Stuttgart, 1991)) or in the textbook by Storhas (Bioreaktoren and periphere Einrichtungen [Bioreactors and peripheral equipment] (Vieweg Verlag, Brunswick/Wiesbaden, 1994)).

The culture medium to be used must suitably meet the requirements of the bacterial strains in question. Descriptions of culture media for various bacteria can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing bacteria usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 20° C. to 45° C. and preferably 25° C. to 40° C. The culture is continued until formation of the desired product is at a maximum. This aim is normally achieved within 10 to 160 hours.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the fatty acids present therein.

According to a further aspect of the invention there is provided a bacterial cell according to the invention for use in the production of protein, ideally recombinant protein.

According to a further aspect of the invention there is provided a method for the manufacture of at least one polypeptide comprising the steps:
i) providing a vessel comprising a cell according to the invention;
ii) providing cell culture conditions which facilitate the growth of a cell culture contained in said vessel; and optionally
iii) isolating said polypeptide from said cell or the surrounding growth medium.

In a preferred method of the invention said polypeptide is recombinant and more ideally still said recombinant polypeptide is a therapeutic polypeptide as herein disclosed.

In an alternative preferred method of the invention said recombinant polypeptide is a bio-catalytic enzyme as herein disclosed.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

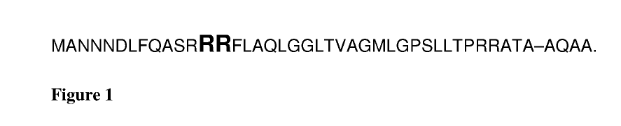

FIG. 1 illustrates the N-terminal sequence of the TorA-GFP construct used in this disclosure. The twin-arginine motif is indicated and AQAA corresponds to the first four residues of the mature TMAO Reductase (TorA) protein;

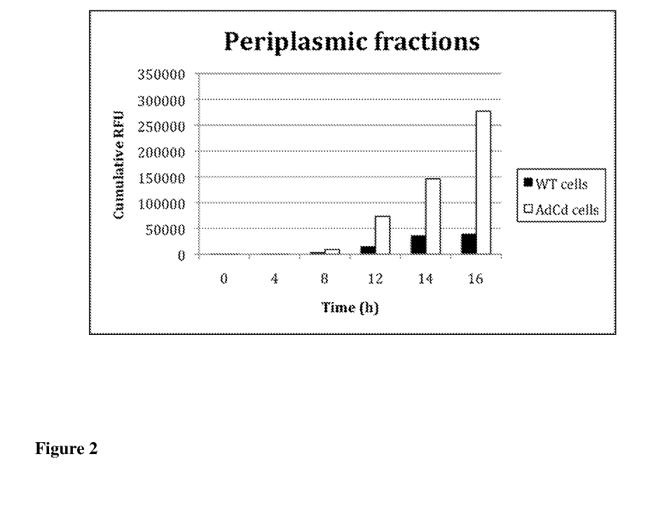

FIG. 2 illustrates the periplasmic distribution of GFP in WT cells (black bars) and E. coli cells over-expressing TatAdCd (TatAdCd pBAD24 cells) (white bars) throughout 16 hours. Equivalent numbers of cells (OD₆₀₀=0.6) were harvested every two hours after induction (Time 0) and periplasmic fractions were assayed for the GFP presence by spectrofluorimetry; data are averages from 5 experiments;

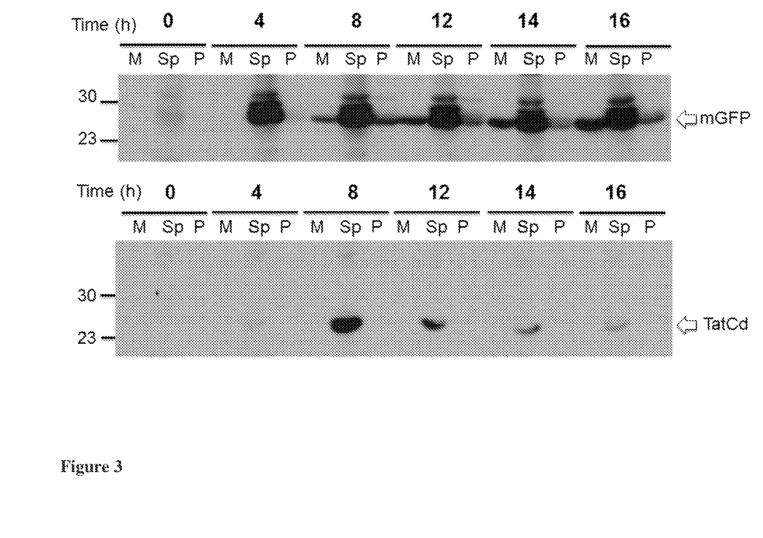

FIG. 3 illustrates the export of TorA-GFP to both the periplasm and medium in E. coli cells expressing TatAdCd. E. coli tat null mutant cells expressing pBAD-AdCd and TorA-GFP were cultured for 16 h after induction with arabinose. At time points indicated, cells were removed and pelleted; samples of the medium were analysed (M) and spheroplasts were generated. Samples of the medium, periplasm (P) and spheroplasts (Sp) were analysed by immunoblotting with antibodies to GFP (upper panel) or the Strep II tag on TatCd (lower panel). mGFP: mature-size GFP. Mobilities of molecular mass markers (in kDa) are shown on the left. The results show that TorA-GFP is exported to the periplasm and processed to the mature size, but there is also a progressive appearance of mature GFP in the medium over time. After 16 h of culture, the majority of exported GFP is in the medium with relatively little found in the periplasm;

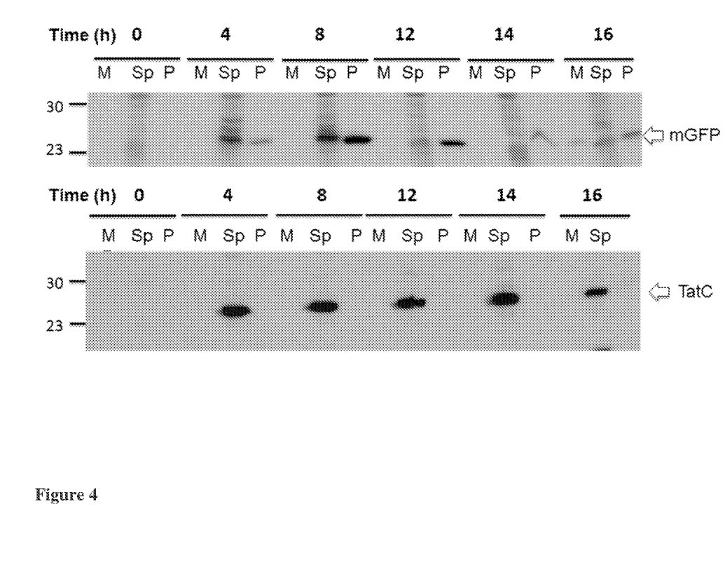

FIG. 4 illustrates E. coli cells overexpressing the natural E. coli TatABC proteins (Tat system) target TorA-GFP to the periplasm but not the medium. E. coli tat null mutant cells expressing pEXT-ABC and TorA-GFP were cultured for 16 h after induction with arabinose. At time points indicated, cells were removed and pelleted; samples of the medium were analysed (M) and spheroplasts were generated. Samples of the periplasm (P) and spheroplasts (Sp) were analysed by immunoblotting with antibodies to GFP (upper panel) or the Strep II tag on TatC (lower panel). mGFP: mature-size GFP. Mobilities of molecular mass markers (in kDa) are shown on the left. The results show that mature size GFP appears in the periplasm but only a very small proportion is found in the medium, even at 16 h;

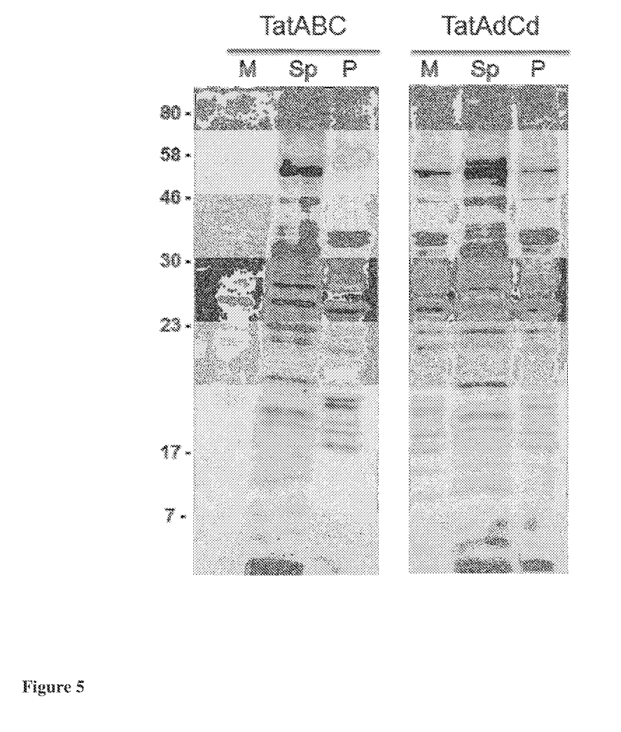

FIG. 5 illustrates TatAdCd-expressing cells release periplasmic contents into the extracellular medium. Samples of the periplasm, spheroplast and medium (P, Sp, M) from 14 h time points of the TatAdCd cultures in FIG. 3, and the TatABC cultures in FIG. 4, were analysed by silver staining of SDS PAGE gels. Mobilities of molecular mass markers (in kDa) are shown on the left. The extracellular medium samples from the TatABC-expressing cells contain very little protein whereas the periplasmic samples from TatAdCd-expressing cells contain more protein than the periplasmic sample. The band patterns of the periplasmic and medium samples are clearly identical, confirming that the periplasmic contents have been partially released into the medium;

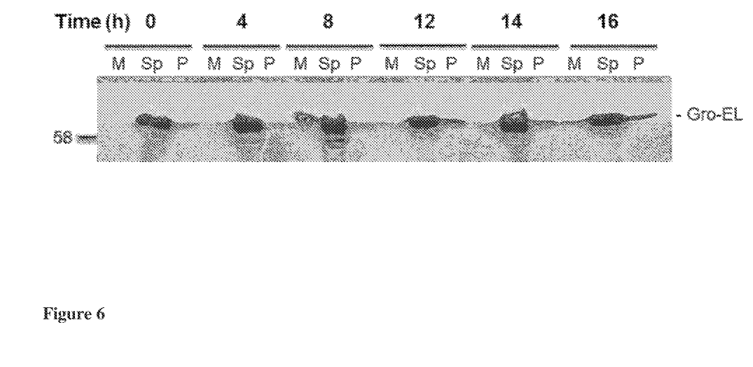

FIG. 6 illustrates the plasma membrane of TatAdCd-expressing cells remains largely intact throughout the fermentation process, and the cells have not lysed. Samples of the medium, spheroplast and periplasm fractions (M, Sp, P) from the TatAdCd-expressing cells shown in FIG. 4 were immunoblotted for GroEL, a marker for the cytoplasmic compartment. At all time points, the majority of the GroEL is found in the spheroplast fraction, with very little found in the periplasm or medium samples. This confirms that little cell lysis has occurred; and

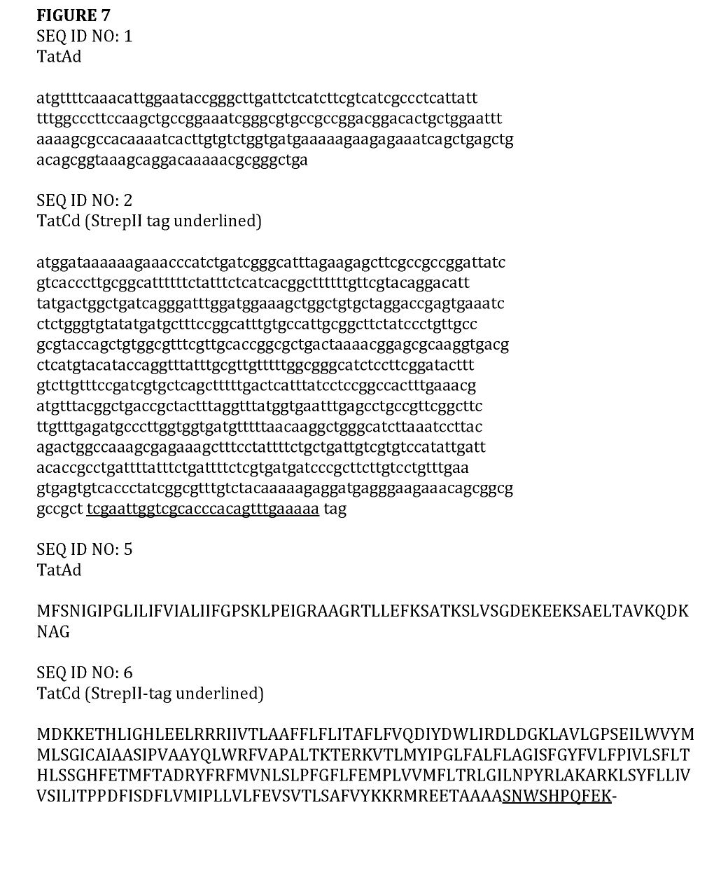

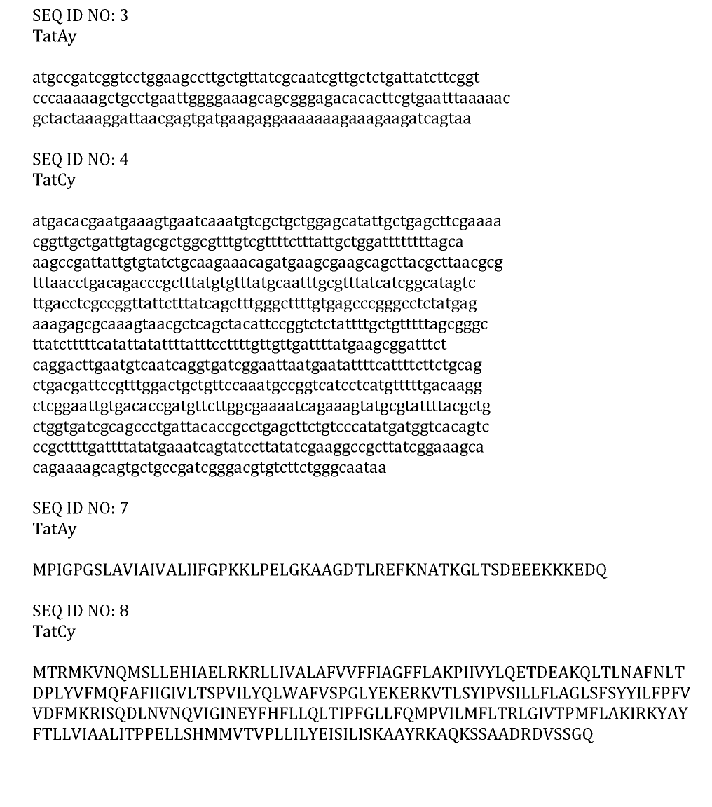

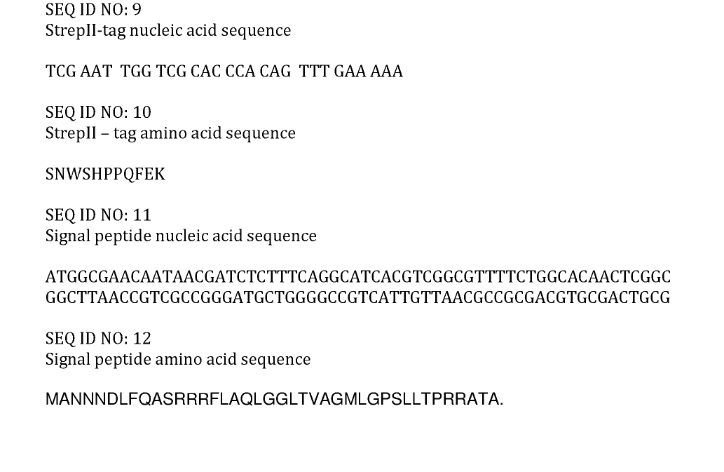

FIG. 7 shows the sequences of the nucleic acid molecules and polypeptides used to work the invention.

### DEFINITIONS

The terms “recombinant polypeptide” or “recombinant protein” is equivalent to “heterologous polypeptide” or “heterologous protein” and includes pharmaceutical or therapeutic polypeptides or proteins; or polypeptides/proteins used for example in bio-catalysis.

Therapeutic polypeptides which are “pharmaceutical polypeptides” (cytokines e.g. growth hormone; leptin; erythropoietin; prolactin; TNF, interleukins (IL), IL-2, IL-3, IL-4, IL-5, IL-6, IL-7, IL-9, IL-10, IL-11; the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin-1 (CT-1); leukemia inhibitory factor (LIF); oncostatin M (OSM); interferons, e.g. interferon α, interferon β, interferon ε, interferon κ and ω interferon are included within the scope of the invention.

Therapeutic polypeptides are also chemokines. The term “chemokine gene” refers to a nucleotide sequence, the expression of which in a cell produces a cytokine. The term chemokine refers to a group of structurally related low-molecular cytokines weight factors secreted by cells that are structurally related having mitogenic, chemotactic or inflammatory activities. They are primarily cationic proteins of 70 to 100 amino acid residues that share four conserved cysteine. These proteins can be sorted into two groups based on the spacing of the two amino-terminal cysteines. In the first group, the two cysteines are separated by a single residue (C-x-C), while in the second group; they are adjacent (C-C). Examples of member of the ‘C-x-C’ chemokines include but are not limited to platelet factor 4 (PF4), platelet basic protein (PBP), interleukin-8 (IL-8), melanoma growth stimulatory activity protein (MGSA), macrophage inflammatory protein 2 (MIP-2), mouse Mig (m119), chicken 9E3 (or pCEF-4), pig alveolar macrophage chemotactic factors I and II (AMCF-I and -II), pre-B cell growth stimulating factor (PBSF), and IP10. Examples of members of the ‘C-C’ group include but are not limited to monocyte chemotactic protein 1 (MCP-1), monocyte chemotactic protein 2 (MCP-2), monocyte chemotactic protein 3 (MCP-3), monocyte chemotactic protein 4 (MCP-4), macrophage inflammatory protein 1 α (MIP-1-α), macrophage inflammatory protein 1 β (MIP-1-β), macrophage inflammatory protein 1-γ (MIP-1-γ), macrophage inflammatory protein 3 α (MIP-3-α, macrophage inflammatory protein 3 β (MIP-3-β), chemokine (ELC), macrophage inflammatory protein-4 (MIP-4), macrophage inflammatory protein 5 (MIP-5), LD78 β, RANTES, SIS-epsilon (p500), thymus and activation-regulated chemokine (TARC), eotaxin, 1-309, human protein HCC-1/NCC-2, human protein HCC-3.

Therapeutic polypeptides which are “anti-angiogenic” polypeptides (e.g. angiostatin, inhibitors of vascular endothelial growth factor (VEGF) such as Tie 2 (as described in PNAS (USA) (1998) 95:8795-8800) and endostatin.

Therapeutic polypeptides include peptide hormones such as GLP-1, anti-diuretic hormone; oxytocin; gonadotropin releasing hormone, corticotrophin releasing hormone; calcitonin, glucagon, amylin, A-type natriuretic hormone, B-type natriuretic hormone, ghrelin, neuropeptide Y, neuropeptide YY₃₋₃₆, growth hormone releasing hormone, somatostatin. Therapeutic polypeptides also includes follicle stimulating hormone (FSH) α subunit, follicle stimulating hormone (FSH) β subunit, luteinizing hormone [LH] β subunit, thyroid stimulating hormone [TSH] β subunit.

Therapeutic polypeptide can also mean an antigenic polypeptide for use in a vaccine. Many modern vaccines are made from protective antigens of the pathogen or disease that are separated by purification or molecular cloning. These vaccines are known as ‘subunit vaccines’. The development of subunit vaccines (e.g. vaccines in which the immunogen is a purified protein) has been the focus of considerable research in recent years. The emergence of new pathogens and the growth of antibiotic resistance have created a need to develop new vaccines and to identify further candidate molecules useful in the development of subunit vaccines. Likewise the discovery of novel vaccine antigens from genomic and proteomic studies is enabling the development of new subunit vaccine candidates, particularly against bacterial pathogens and cancers.

Also included within the scope of therapeutic polypeptides are therapeutic antibodies and antibody fragments. Preferably said antibodies are monoclonal antibodies or at least the active binding fragments thereof. Therapeutic antibodies may be antibodies which bind and inhibit the activity of biological molecules, e.g. ligands or receptors. Monoclonal antibodies may be humanised or chimeric antibodies.

Preferably said fragments are single chain antibody variable regions (scFV's) or domain antibodies. If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety. A modified antibody, or variant antibody, and reference antibody, may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan. Most highly preferred are variants which show enhanced biological activity.

A therapeutic polypeptide is an enzyme, for example a therapeutic enzyme selected from the group tissue plasminogen activator, activated protein C, deoxyribonuclease I, β glucocerebrosidase and α galactosidase, adenosine deaminase, arginine deiminase, urate oxidase, L asparaginase, factor VIIa, factor IX, α Liduronidase, urostreptokinase, staphylokinases, ancrodkinase, acid α glucosidase, superoxide dismutase hyaluronidase, lactase, pancreatin, α galactosidase, galsulfase, idursulfase, asparaginase, lipase, uricase, methioninase, streptokinase, superoxide dismutase and α-chymotrypsin.

Enzymes used in bio-catalysis are also within the scope of the invention and are not typically therapeutic polypeptides. For example, the production of liquid biofuels the cellulose and other polysaccharides in the biomass must be converted to sugars by saccharification. Saccharification is a process by which plant lignocellulosic materials (e.g., lignin, cellulose, hemicellulose) are hydrolysed to glucose through chemical and enzymatic means. Typically this involves the pre-treatment of plant material with alkali to remove lignin followed by enzyme digestion of cellulose. The enzymes currently available for industrial lignocellulose saccharification involve a cocktail of endoglucanases, cellobiohydrolases and glucosyl hydrolases.

Other bio-catalytic enzymes include restriction endonucleases, DNases, RNases, DNA and RNA polymerases including thermostable DNA polymerases; and proteases.

### Materials and Methods

### Media

Luria-Bertani (LB) broth (10 g/L bacto-tryptone, 5 g/L yest extract, 10 g/L sodium chloride) was used for the aerobic growth of E. coli in liquid media. Antibiotics were added, when necessary, to the following concentrations: ampicillin 100 μg/mL, kanamycin 50 μg/mL. LB medium with 16% (w/v) agar was used for the aerobic growth of E. coli on plates.

The defined medium recipe was based on that described by Garcia-Arrazola et al., (2005) and was supplemented with ampicillin (100 μg/mL) and kanamycin (50 μg/mL) where appropriate.

### Plasmid and Strain Constructions

Methods for cloning and transformation are described, for example, in the Sambrook et al., 2001, Molecular Cloning: A Laboratory Manual (3rd Ed.), Cold Spring Harbor Press, N.Y.

All strains and plasmids used in the following examples are listed in Table 1.

| | | Reference |
|---|---|---|
| Strain | Genotype | |
| MC4100 | F ΔlacU169 araD139 rpsL150 | Casadaban & |
| | relA1 ptsF rbs flbB5301 | Cohen, 1979 |
| ΔtatABCDE | MC4100; ΔtatABCDE, Ara^{r} | Bolhuis et al., 2000 |
| Plasmid | Details | |
| pEXT22 | IPTG inducible cloning vector | Dykxhoorn et al., 1996 |
| | lacl kan^{r} | |
| TorA-GFP | pEXT22, TorA-GFP | this study |
| pAdCds | pBAD24, tatAd tatCd | Barnett et al., 2008, this |
| | | Strep II ™ tag study |
| pJDT1 | pBAD24, TorA-GFP | Thomas e al; 2001 |
| pEXT-ABC | pEXT22, tatA, tatB, tatC | Matos et al., 2009 |
| | StrepII ™ tag | |

B. subtilis TatAdCd was expressed using pBAD-AdCd (Barnett et al., 2008). The TatAdCd operon was modified by introduction of a C-terminal Strep II-tag for immunodetection. The substrate used in this study, TorA-GFP was described by Thomas et al. (2001). Briefly, cDNA encoding the complete amino acid sequence (amino acids 1 to 44) of the E. coli Tat-dependent TorA signal sequence plus the first four residues of the mature TorA in frame was fused to the 5′ end of the gene encoding green fluorescent protein (gfp mut3* version) downstream from a pBAD promoter in pBAD24 vector.

This TorA-GFP fusion was cloned into the pEXT22 vector (Dykxhoorn et al., 1996) under the control of IPTG-inducible pTac promoter.

### Fermentation

Starter cultures were first grown in 200 mL of Luria-Bertani broth supplemented with antibiotics in 1 Litre shake-flasks for 3 hours, 30° C. Next, 10% (v/v) of the culture was transferred to 1 Litre shake-flasks containing 200 mL of defined medium with antibiotic supplementation and incubated for 20 hours, 30° C. Samples of OD₆₀₀=0.3 of each defined medium shake-flasks were used to inoculate fresh defined medium to a final volume of 900 mL in Infors Multifors 1 Litre Fermenters (Infors UK Ltd, Reigate, UK). For induction of protein expression, cells were induced at the point of fermentation inoculation only with 100 μM arabinose and 1 mM IPTG. Samples were taken two hourly for OD₆₀₀ readings, dry cell weight measurements and fractionation experiments. The culture was maintained at 30° C. throughout the fermentation, and pH was maintained at 6.95 using 5% (v/v) H₂SO₄ and 5% (v/v) ammonia solution.

Cell density was measured by optical density in the range of 0.1-1.0 at 600 nm. Dry cell weight quantification (DCW) was performed by collecting 1 mL of culture sample in duplicate in pre-weighted, pre-dried 1.5 mL polypropylene tubes. Cells pellets were dried for 24 hours at 100° C. and tubes weighted.

Fractionation of E. coli Cells

Cells were separated into periplasmic, cytoplasmic and membrane fractions using a procedure on the EDTA/lysozyme/cold osmotic shock method (Randall & Hardy, 1986). Typically cells were centrifuged (4 min at 17 000 g) supernatant was removed and filtered. Harvested cells were resuspended in 1 mL chilled buffer containing 100 mM Tris-acetate pH 8.2, 0.5M sucrose, 5 mM EDTA. 40 μL lysozyme (2 mg/mL) and 500 μL ice cold H₂O was added before incubation on ice for 5 min followed by the addition of 5 mM MgSO₄. The spheroplasts were pelleted by centrifugation at 20,800×g (Eppendorf 5417R) and the supernatant was collected as the periplasmic fraction. Spheroplasts were washed in 1 mL chilled buffer containing 50 mM Tris-acetate pH 8.2, 0.25 mM sucrose, 10 mM MgSO₄ and pelleted by centrifugation. The supernatant was discarded and the spheroplasts were resuspended in 1 mL chilled buffer containing 50 mM Tris-acetate pH 8.2, 2.5 mM EDTA. Spheroplasts were lysed by sonication and membranes were separated from the cytoplasmic fraction by centrifugation at 265,000×g (Beckman TL100, TLA 100.3 rotor) for 30 min at 4° C. The membranes formed a pellet and the supernatant was collected as the cytoplasmic fraction. Membranes were solubilised by resuspending in 500 μL detergent containing buffer. Membranes were incubated at 4° C. with constant rotation for 16 hours. Insoluble material was removed by centrifugation at 265,000×g for 15 min at 4° C.

### Protein Separation

SDS polyacrylamide gels were cast and run on a vertical gel electrophoresis system (CBS) according to manufacturer's instructions. Typically, 0.75 mm gels were prepared with a separating gel (15% Protogel acrylamide solution, 375 mM Tris-HCl, pH 8.8, 0.1% SDS, 0.02% APS, 0.06% TEMED) and a stacking gel (5% Protogel acrylamide solution, 125 mM Tris-Hcl pH 6.8, 0.1% SDS, 0.6% APS, 0.06% TEMED). Samples were prepared by mixing with SDS sample loading buffer (125 mM Tris-HCl pH 6.8, 20% glycerol, 4% SDS, 0.02% bromophenol blue, 5% β-mercaptoethanol) and boiling at 50° C. for 10 min.

### Detection of Proteins by Immunoblotting

After transfer, PVDF membranes to be immunoblotted with antibodies to the StrepII™ tag were blocked with PBS-T containing 3% BSA for at least 1 hour. The membranes were washed in PBS-T, before incubation in PBS-T with 6 μg/mL avidin for 10 min. The Streptactin-horseradish peroxidise (HRP) conjugate antibody (IBA) was added directly to the avidin solution according to the manufacturer's instructions and incubated, with agitation, for 2 hours.

PVDF membranes to be immunoblotted with GFP and GroEL antibodies were blocked with PBS-T containing 5% (w/v) dried skimmed milk powder for at least 1 hour. The membranes were washed in PBS-T before incubation with PBS-T containing the desired primary antibody for 1 hour. The membranes were washed before incubation with the secondary antibody for 1 hour. The membranes were washed and immunoreactive bands were detected using ECL kit (Amersham Biosciences) according to the manufacturer's instructions. X-ray films (Super RX film, Fujifilm) were developed on an AGFA Curix 60 automatic developer according to the manufacturer's instructions.

TABLE 2
Antibodies used in this study

| Antibody | Concentration | Manufacturer/Source |
|---|---|---|
| Streptactin-HRP conjugate | 1 in 12000 | IBA |
| anti-GFP | 1 in 10000 | Invitrogen |
| anti-GroEL | 1 in 10000 | Sigma |
| anti-rabbit IgG (H + L), HRP | 1 in 10000 | Promega |
| conjugate | | |

### EXAMPLE 1

### Experimental Design and Constructs Used in this Study

It has previously been shown that GFP can be exported to the periplasm in E. coli if a Tat signal peptide is present at the N-terminus (Thomas et al., 2001). It has also been shown that the B. subtilis TatAdCd system is active in an E. coli tat null mutant, and able to export both endogenous E. coli Tat substrates and Tat signal peptide-GFP constructs to the periplasm (Barnett et al., 2008; Mendel et al., 2008). These experiments were carried out in shake-flask cultures, and in this study we sought to compare the export capacities of the E. coli and B. subtilis Tat systems when grown in fermentation conditions. The substrate used comprised the twin-arginine (Tat) signal peptide of TMAO reductase (TorA) fused to GFP (specifically, the GFPmut3* version). The construct is termed TorA-GFP, and this protein has been shown to be exported to the periplasm in tat null mutant cells expressing TatAdCd (Barnett et al., 2008).

In this study we used two different plasmids to co-express TorA-GFP together with either the B. subtilis TatAdCd proteins or the E. coli TatABC proteins: the IPTG-inducible pEXT22 vector (Dykxhoorn et al., 1996). and the arabinose-inducible pBAD24 plasmid (Guzman et al., 1995). Preliminary data (not shown) indicated that the highest export efficiencies were obtained when TatAdCd was expressed from pBAD24 and the TorA-GFP substrate was expressed from pEXT22. For unknown reasons, the over-expressed E. coli TatABC gave best results when expressed form pEXT22.

### EXAMPLE 2

### E. coli Over-Expressing TatAdCd Accumulates and Exports Greater Amounts of GFP into the Medium

We first tested whether over-expression of TatAdCd in this strain enabled TorA-GFP to be exported more efficiently than in wild type E. coli cells in batch fermentations. TorA-GFP was expressed using pEXT22 (as indicated above) and TatAdCd were overexpressed using the pBAD24 plasmid (the construct is termed pBAD-AdCd). Cells were grown for 16 h and, to study the subcellular distribution of the active GFP, equivalent numbers of cells (OD₆₀₀=0.6) were harvested every 2 hours after induction (Time 0) and lysed by sonication. This was followed by quantification of GFP using spectrofluorimetry and immunoblot assays, and the levels of periplasmic GFP from each culture are shown in FIG. 2. The data show that the periplasmic GFP accumulates steadily, and that TatAdCd-expressing cells export much greater levels of GFP than do wild type cells. This confirms that the TatAdCd is active and able to sustain export throughout the 16-h fermentation process.

### EXAMPLE 3

### E. coli Cells Over-Expressing TatAdCd Allow Leakage of Periplasmic GFP into the Medium

We then analysed the TatAdCd-expressing cells in more detail to assess the export efficiency throughout the fermentation process. FIG. 3 shows data for cells expressing pBAD-AdCd and TorA-GFP. At each time point, cells were fractionated to yield a periplasmic sample (P) and a spheroplast (Sp) sample representing the remaining cytoplasm and membrane fractions. Samples of the medium were also analysed (M) by pelleting the bacteria before spheroplasting and analysing the supernatant. The samples were immunblotted with antibodies to GFP (upper panel) to test for export efficiency, and to TatCd (lower panel) to assess the expression of the TatCd component of the translocase. The upper blot shows that GFP is detected 4 h after induction, at which point it is mainly in the spheroplast fraction. Thereafter, the periplasmic protein becomes more prominent, but surprisingly, mature-size GFP is found in the medium (M) at even greater levels. This finding is unexpected because in most studies the E. coli outer membrane is not sufficiently leaky to allow periplasmic proteins into the medium.

The TatCd blot (lower panel) shows that high levels of TatCd are found in the spheroplast fraction at the 8 h time point, after which the levels drop. The TatCd is present in the plasma membrane as shown by Barnett et al. (2008) and immunoblotting of membranes samples from these cells (data not shown). Overall, the results show that TatAdCd supports efficient export of TorA-GFP, with the unexpected result that protein is released into the medium. It is notable that there is significant accumulation of TorA-GFP in the spheroplasts, and this might be due to saturation of the TatAdCd export machinery since the TorA-GFP is synthesised in large quantities.

### EXAMPLE 4

### E. coli Cells Over-Expressing TatABC and TorA-GFP Retain Exported GFP in the Periplasm

The above data show that cells expressing TatAdCd and TorA-GFP release mature GFP into the medium. This trait is unexpected, and it could be due, however indirectly, to: (i) over-expression of the Tat machinery, (ii) over-expression of the TorA-GFP substrate or (iii) the use of fermentation conditions to analyse export (most studies on the Tat system have been carried out in shake flask cultures over much shorter time scales). We therefore carried out a similar fermentation run in which E. coli TatABC was over-expressed instead of TatAdCd; this would directly test whether it is the over-expressed TatAdCd that causes the release of GFP. In these tests, TatABC were over-expressed using pEXT22 and TorA-GFP was expressed using pBAD24 (as explained above). However, similar results were obtained when the TatABC and TorA-GFP were expressed from pBAD24 and pEXT22, respectively (data not shown).

The data for cells expressing TatABC and TorA-GFP are shown in FIG. 4. The GFP blot (upper panel) shows the appearance of TorA-GFP at the 4 h time point, and mature-size GFP is found in the periplasm at this and subsequent times, confirming export. It is interesting that the peak periplasmic GFP signal is observed at the 8 h time point, with levels declining at later times, and we speculate that this reflects the induction of proteases that may remove GFP in the periplasmic fraction. Importantly, the amount of GFP found in the medium is very low, amounting to only a very small percentage (less than 5%) of the total exported GFP even at later time points. The lower panel shows a blot for TatC, which indicates the presence of this Tat component over the entire fermentation run. In summary, the over-expressed TatABC supports export of TorA-GFP to the periplasm, with very little evidence of release of GFP into the medium. In this respect, the cells appear to differ dramatically from those expressing TatAdCd.

### EXAMPLE 5

### E. coli Over-Expressing TatAdCd Leaks GFP into Medium

The presence of GFP in the medium of TatAdCd-expressing cells raises an important question: is the GFP being selectively released through the outer membrane, or is the outer membrane leaky enough to allow the entire periplasmic protein complement into the medium? We addressed this in two ways. In the first (FIG. 5) we analysed Coomassie-stained gels of the periplasm, medium and spheroplast fractions from these cells and also TatABC/TorA-GFP-expressing cells. The data confirm the near-absence of proteins in the medium from TatABC-expressing cells, consistent with the finding that very little GFP is found in this fraction. In contrast, the medium from the TatAdCd culture shows a pattern of bands that is essentially identical to that present in the periplasmic fraction. The band pattern is clearly different to that of the spheroplasts.

The second test is shown in FIG. 6. Here, we immunoblotted the periplasm, medium and spheroplast fractions from a TatAdCd fermentation with antibodies to GroEL, a known cytoplasmic folding chaperone that is often used as a marker for this compartment. The data show that both the periplasmic and medium samples contain very little of the GroEl, which is almost exclusively found in the spheroplasts. The combined data indicate that the TatAdCd-expressing cells export TorA-GFP into the periplasm, and that the outer membrane becomes leaky during the course of the fermentation run with the result that the bulk periplasmic protein is released into the medium.

### REFERENCES

Barnett J P, Eijlander R T, Kuipers O P, Robinson C (2008) A minimal Tat system from a gram-positive organism: a bifunctional TatA subunit participates in discrete TatAC and TatA complexes. J Biol Chem 283, 2534-2542
Bolhuis A, Bogsch E G, Robinson C (2000) Subunit interactions in the twin-arginine translocase complex of Escherichia coli. FEBS Letts 472: 88-92
Casadaban M J, Cohen S N (1979) Lactose genes fused to exogenous promoters in one step using a Mu-lac bacteriophage: in vivo probe for transcriptional control sequences. Proc Natl Acad Sci USA 76: 4530-4533
Dykxhoorn D M, St. Pierre R, Linn T. 1996. A set of compatible tac promoter expression vectors. Gene 177, 133-136.)
García-Arrazola R, Chau Siu S, Chan G, Buchanan I, Doyle B, Titchener-Hooker N, Baganz F (2005) Evaluation of a pH-stat feeding strategy on the production and recovery of Fab′ fragments from E. coli. Biochem Eng J 23: 221-230
Guzman, L. M., Belin, D., Carson, M. J. and Beckwith., J. (1995). Tight regulation, modulation, and high-level expression by vectors containing the arabinose PBAD promoter. J. Bacteriol. 177, 4121-4130.
Matos, CFRO., Di Cola, A and Robinson, C. (2009). TatD is a central component of a Tat translocon-mediated quality control system for exported FeS proteins in Escherichia coli. EMBO Rep. 10, 474-479.
Mendel, S., McCarthy, A., Barnett, J. P., Eijlander, R. T., Kuipers, O. P. and Robinson, C. (2008). The Escherichia coli TatABC system and a Bacillus subtilis TatAC-type system recognize three distinct targeting determinants in twin-arginine signal peptides. J. Mol. Biol. 375, 661-672.
Müller M, Klosgen R B. (2005). The Tat pathway in bacteria and chloroplasts (review). Mol Membr Biol. 22:113-21.
Randall L L, Hardy S L S (1986) Correlation of competence for export with lack of tertiary structure of the mature species: a study in vivo of maltose-binding in E. coli. Cell 46: 921-928
Robinson C, Bolhuis A. (2004). Tat-dependent protein targeting in prokaryotes and chloroplasts. Biochim Biophys Acta. 1694:135-47.
Thomas, J D, Daniel, R A, Errington, J, Robinson, C. (2001). Export of active green fluorescent protein to the periplasm by the twin arginine translocase (Tat) pathway in Escherichia coli. Mol Microbiol 39, 47-53.
Sambrook et al., (2001) Molecular Cloning: A Laboratory manual (3^{rd} Ed.) Cold Spring Harbor Press, N.Y.
Wexler M, Sargent F, Jack R L, Stanley N R, Bogsch E G, Robinson C, Berks B C, Palmer T (2000) TatD Is a cytoplasmic protein with DNase activity. No requirement for TatD family proteins in Sec-independent protein export. J Biol Chem 275: 16717-16722

1. A Gram-negative bacterial cell wherein said cell is genetically modified by transformation with a nucleic acid molecule encoding a polypeptide complex comprising a twin arginine translocase [Tat] system isolated or made from a Gram positive bacterial species and further wherein said Gram negative bacterial cell expresses a polypeptide which polypeptide is adapted to interact with said Tat complex and is secreted into the cell culture medium.

2. The cell according to claim 1 wherein said polypeptide is recombinant.

3. The cell according to claim 1 wherein said Tat system is over-expressed when compared to a non-transformed reference bacterial cell of the same species expressing a reference protein.

4. The cell according to claim 1 wherein said Tat system comprises a polypeptide complex TatAdCd.

5. The cell according to claim 1 wherein said Tat system comprises a polypeptide complex TatAyCy.

6. The cell according to any one of claim 1 wherein said polypeptide is adapted by the provision of a signal peptide that interacts with the Tat system.

7. The cell according to claim 6 wherein said signal peptide comprises a twin arginine motif sequence, a hydrophobic domain and a consensus amino acid sequence for a peptidase.

8. The cell according to claim 7 wherein said twin arginine motif comprises the amino acid sequence RRxFL (SEQ ID NO: 14) wherein x is any amino acid residue.

9. The cell according to claim 8 wherein said signal peptide comprises an amino acid sequence: MANNNDLFQASRRRFLAQLGGLTVAGMLGPSLLTPRRATA (SEQ ID NO:12).

10. The cell according to claim 8 wherein said signal peptide consists essentially of the amino acid sequence: MANNNDLFQASRRRFLAQLGGLTVAGMLGPSLLTPRRATA (SEQ ID NO:12).

11. The cell according to claim 1 wherein said nucleic acid molecule is selected from the group consisting of:
i) a nucleotide sequence as represented by SEQ ID NOs: 1 and 2 or SEQ ID NOs: 3 and 4;
ii) a nucleotide sequence wherein said sequence is degenerate as a result of the genetic code to the nucleotide sequence defined in (i);
iii) a nucleic acid molecule, the complementary strand of which hybridizes under stringent hybridization conditions to the nucleic acid sequences in i) and ii) above wherein said nucleic acid molecule encodes a twin arginine translocase;
iv) a nucleotide sequence that encodes a twin arginine translocase comprising an amino acid sequence represented by SEQ ID NOs: 5 and 6 or SEQ ID NOs: 7 and 8; and
v) a nucleotide sequence that encodes a polypeptide comprising an amino acid sequence wherein said amino acid sequence is modified by addition, deletion, or substitution of at least one amino acid residue as represented in iv) above and which has retained or enhanced twin arginine translocase activity.

12. The cell according to claim 11 wherein said nucleic acid molecule comprises or consists of a nucleotide sequence as represented in SEQ ID NOs: 1 and 2 or SEQ ID NOs: 3 and 4.

13. The cell according to claim 11 wherein said nucleic acid molecule encodes a polypeptide comprising the amino acid sequence in SEQ ID NOs: 5 and 6 or SEQ ID NOs: 7 and 8.

14. The cell according to claim 1, wherein said Gram-negative bacterial cell is Escherichia coli.

15. A bacterial cell culture comprising a bacterial cell according to claim 1.

16. A cell culture vessel comprising a bacterial cell culture according to claim 15.

17. The vessel according to claim 16 wherein said cell culture vessel is a fermentor.

18-19. (canceled)

20. A method for the manufacture of at least one polypeptide comprising the steps:
i) providing a vessel comprising a cell according to claim 1 and a growth medium;
ii) growing said cell under cell culture conditions which facilitate the growth of said cell; and optionally
iii) isolating said recombinant polypeptide from said cell or the growth medium.

21. The method according to claim 20 wherein said polypeptide is a recombinant polypeptide.

22. The method according to claim 20 wherein said polypeptide is a therapeutic polypeptide.

23. The method according to claim 20 wherein said polypeptide is a bio-catalytic enzyme.

24. A method for the production of at least one polypeptide or protein, comprising:
i) providing a Gram-negative bacterial cell according to claim 1;
ii) growing said bacterial cell in a growth medium under cell culture conditions which facilitate the growth of said bacterial cell; and optionally
iii) isolating said recombinant polypeptide from said bacterial cell and/or from the growth medium.

25. The method according to claim 24 wherein said polypeptide is a recombinant polypeptide.


SEQUENCE LISTING (13 sequences)

SEQ ID NO: 1 - DNA, 213 bp, Escherichia coli
atgttttcaaacattggaataccgggcttgattctcatcttcgtcatcgccctcattatttttggcccttccaagctgccggaaatcgggcgtgccgccggacggacactgctggaatttaaaagcgccacaaaatcacttgtgtctggtgatgaaaaagaagagaaatcagctgagctgacagcggtaaagcaggacaaaaacgcgggctga

SEQ ID NO: 2 - DNA, 759 bp, Escherichia coli
atggataaaaaagaaacccatctgatcgggcatttagaagagcttcgccgccggattatcgtcacccttgcggcattttttctatttctcatcacggcttttttgttcgtacaggacatttatgactggctgatcagggatttggatggaaagctggctgtgctaggaccgagtgaaatcctctgggtgtatatgatgctttccggcatttgtgccattgcggcttctatccctgttgccgcgtaccagctgtggcgtttcgttgcaccggcgctgactaaaacggagcgcaaggtgacgctcatgtacataccaggtttatttgcgttgtttttggcgggcatctccttcggatactttgtcttgtttccgatcgtgctcagctttttgactcatttatcctccggccactttgaaacgatgtttacggctgaccgctactttaggtttatggtgaatttgagcctgccgttcggcttcttgtttgagatgcccttggtggtgatgtttttaacaaggctgggcatcttaaatccttacagactggccaaagcgagaaagctttcctattttctgctgattgtcgtgtccatattgattacaccgcctgattttatttctgattttctcgtgatgatcccgcttcttgtcctgtttgaagtgagtgtcaccctatcggcgtttgtctacaaaaagaggatgagggaagaaacagcggcggccgcttcgaattggtcgcacccacagtttgaaaaatag

SEQ ID NO: 3 - DNA, 174 bp, Escherichia coli
atgccgatcggtcctggaagccttgctgttatcgcaatcgttgctctgattatcttcggtcccaaaaagctgcctgaattggggaaagcagcgggagacacacttcgtgaatttaaaaacgctactaaaggattaacgagtgatgaagaggaaaaaaagaaagaagatcagtaa

SEQ ID NO: 4 - DNA, 765 bp, Escherichia coli
atgacacgaatgaaagtgaatcaaatgtcgctgctggagcatattgctgagcttcgaaaacggttgctgattgtagcgctggcgtttgtcgttttctttattgctggattttttttagcaaagccgattattgtgtatctgcaagaaacagatgaagcgaagcagcttacgcttaacgcgtttaacctgacagacccgctttatgtgtttatgcaatttgcgtttatcatcggcatagtcttgacctcgccggttattctttatcagctttgggcttttgtgagcccgggcctctatgagaaagagcgcaaagtaacgctcagctacattccggtctctattttgctgtttttagcgggcttatctttttcatattatattttatttccttttgttgttgattttatgaagcggatttctcaggacttgaatgtcaatcaggtgatcggaattaatgaatattttcattttcttctgcagctgacgattccgtttggactgctgttccaaatgccggtcatcctcatgtttttgacaaggctcggaattgtgacaccgatgttcttggcgaaaatcagaaagtatgcgtattttacgctgctggtgatcgcagccctgattacaccgcctgagcttctgtcccatatgatggtcacagtcccgcttttgattttatatgaaatcagtatccttatatcgaaggccgcttatcggaaagcacagaaaagcagtgctgccgatcgggacgtgtcttctgggcaataa

SEQ ID NO: 5 - protein, 70 aa, Escherichia coli
MFSNIGIPGLILIFVIALIIFGPSKLPEIGRAAGRTLLEFKSATKSLVSGDEKEEKSAELTAVKQDKNAG

SEQ ID NO: 6 - protein, 252 aa, Escherichia coli
MDKKETHLIGHLEELRRRIIVTLAAFFLFLITAFLFVQDIYDWLIRDLDGKLAVLGPSEILWVYMMLSGICAIAASIPVAAYQLWRFVAPALTKTERKVTLMYIPGLFALFLAGISFGYFVLFPIVLSFLTHLSSGHFETMFTADRYFRFMVNLSLPFGFLFEMPLVVMFLTRLGILNPYRLAKARKLSYFLLIVVSILITPPDFISDFLVMIPLLVLFEVSVTLSAFVYKKRMREETAAAASNWSHPQFEK

SEQ ID NO: 7 - protein, 57 aa, Escherichia coli
MPIGPGSLAVIAIVALIIFGPKKLPELGKAAGDTLREFKNATKGLTSDEEEKKKEDQ

SEQ ID NO: 8 - protein, 254 aa, Escherichia coli
MTRMKVNQMSLLEHIAELRKRLLIVALAFVVFFIAGFFLAKPIIVYLQETDEAKQLTLNAFNLTDPLYVFMQFAFIIGIVLTSPVILYQLWAFVSPGLYEKERKVTLSYIPVSILLFLAGLSFSYYILFPFVVDFMKRISQDLNVNQVIGINEYFHFLLQLTIPFGLLFQMPVILMFLTRLGIVTPMFLAKIRKYAYFTLLVIAALITPPELLSHMMVTVPLLILYEISILISKAAYRKAQKSSAADRDVSSGQ

SEQ ID NO: 9 - DNA, 30 bp, Escherichia coli
tcgaattggtcgcacccacagtttgaaaaa

SEQ ID NO: 10 - protein, 11 aa, Escherichia coli
SNWSHPPQFEK

SEQ ID NO: 11 - DNA, 120 bp, Escherichia coli
atggcgaacaataacgatctctttcaggcatcacgtcggcgttttctggcacaactcggcggcttaaccgtcgccgggatgctggggccgtcattgttaacgccgcgacgtgcgactgcg

SEQ ID NO: 12 - protein, 40 aa, Escherichia coli
MANNNDLFQASRRRFLAQLGGLTVAGMLGPSLLTPRRATA

SEQ ID NO: 13 - protein, 44 aa, Escherichia coli
MANNNDLFQASRRRFLAQLGGLTVAGMLGPSLLTPRRATAAQAA